(19) United States
(12) Patent Application Publication
(10) Pub. No.: US 2017/0105704 A1
(43) Pub. Date: Apr. 20, 2017

(54) ULTRASONIC DIAGNOSTIC APPARATUS AND MEDICAL IMAGE PROCESSING APPARATUS

(71) Applicant: Toshiba Medical Systems Corporation, Otawara-shi (JP)

(72) Inventor: Mitsuo AKIYAMA, Otawara (JP)

(73) Assignee: Toshiba Medical Systems Corporation, Otawara-shi (JP)

(21) Appl. No.: 15/196,176

(22) Filed: Jun. 29, 2016

(30) Foreign Application Priority Data
Oct. 15, 2015 (JP) 2015-203757

(51) Int. Cl. A61B 8/08; A61B 8/02; A61B 8/00

(52) U.S. Cl. CPC A61B 8/5207; A61B 8/461; A61B 8/5223; A61B 8/488; A61B 8/486; A61B 8/02; A61B 8/4483

(57) ABSTRACT

An ultrasonic diagnostic apparatus according to a present embodiment includes transceiver circuitry, image generating circuitry and processing circuitry. The transceiver circuitry is configured to transmit, to an ultrasonic probe, a transmission signal for transmitting ultrasonic waves, and to receive a received signal based on the ultrasonic waves. The image generating circuitry is configured to generate an ultrasonic image where temporal variation over a given time period is imaged, based on the received signal, and to display the ultrasonic image to a display. The processing circuitry is configured to: (A) set a measurement range on the ultrasonic image; (B) generate a marker for dividing the measurement range, at substantially equal intervals, the number of the marker according to the numerically number of beats; and (C) superimpose the marker on the displayed ultrasonic image.

### CROSS-REFERENCE TO RELATED APPLICATION

This application is based upon and claims the benefit of priority from Japanese Patent Application No. 2015-203757, filed on Oct. 15, 2015, the entire contents of which are incorporated herein by reference.

### FIELD

An embodiment as an aspect of the present invention relates to an ultrasonic diagnostic apparatus and a medical image processing apparatus.

### BACKGROUND

In the medical field, ultrasonic diagnostic apparatuses are used. The apparatus utilizes ultrasonic waves that are generated using transducers (piezoelectric transducers) of an ultrasonic probe, and images the inside of an object. The ultrasonic diagnostic apparatus causes the ultrasonic probe connected to the ultrasonic diagnostic apparatus to transmit ultrasonic waves into the object to generate a received signal based on reflected waves, and obtains desired ultrasonic images through image processing.

The ultrasonic images include, for example: a B-mode image, which is sectional information at a given time; an M-mode image, in which temporal variation over a given time period is imaged; and a Doppler spectrum image. At the position of a sample volume (SV) that is set on a B-mode image, a Doppler spectrum image is obtained that represents flow velocities (flow velocity transition) obtained over a given time period.

When a heart rate and the like are measured using a Doppler spectrum image, two times (a start time and an end time) for determining a measurement range are set on the Doppler spectrum image. These two times are set at a telediastolic point in a cardiac sequence. The heart rate is calculated from the time interval between the two times and the number of beats in the interval. The result is numerically represented as a heart rate of 84 beats per minute.

For example, in the case where two times including three beats are set and where the interval between the two times, i.e., the measurement range, is 2.5 seconds, the heart rate is (3 [beat]/2.5 [sec.])×60 [sec.]=72 [bpm]. The number of beats is numerically set irrespective of the measurement range, and is preset as a default or set through designation by an operator to change the default value.

The problem the present invention intends to solve is actually solved by providing an ultrasonic diagnostic apparatus and a medical image processing apparatus that allow an operator to intuitively, visually identify an erroneous setting on the number of beats and the measurement range and reduce the test time.

### BRIEF DESCRIPTION OF THE DRAWINGS

In accompanying drawings,

FIG. 1 is a schematic diagram showing a configuration of an ultrasonic diagnostic apparatus according to a present embodiment;

FIG. 2 is a block diagram showing functions of the ultrasonic diagnostic apparatus 10 according to the present embodiment;

FIG. 3 is a diagram to illustrate an example of dividing a measurement range;

FIG. 4 is a diagram to illustrate an example of dividing a measurement range;

FIG. 5 is a diagram to illustrate an example of dividing a measurement range;

FIG. 6 is a diagram to illustrate an example of dividing a measurement range;

FIG. 7 is a diagram to illustrate an example of dividing a measurement range;

FIG. 8 is a flowchart showing operations of the ultrasonic diagnostic apparatus according to the present embodiment;

FIG. 9 is a schematic diagram showing a configuration of a medical image processing apparatus according to a present embodiment; and

FIG. 10 is a block diagram showing functions of the medical image processing apparatus according to the present embodiment.

### DETAILED DESCRIPTION

An ultrasonic diagnostic apparatus and a medical image processing apparatus according to a present embodiment are described with reference to the accompanying drawings.

The ultrasonic diagnostic apparatus according to the present embodiment includes transceiver circuitry, image generating circuitry and processing circuitry. The transceiver circuitry is configured to transmit, to an ultrasonic probe, a transmission signal for transmitting ultrasonic waves, and to receive a received signal based on the ultrasonic waves. The image generating circuitry is configured to generate an ultrasonic image where temporal variation over a given time period is imaged, based on the received signal, and to display the ultrasonic image to a display. The processing circuitry is configured to: (A) set a measurement range on the ultrasonic image; (B) generate a marker for dividing the measurement range, at substantially equal intervals, the number of the marker according to the numerically number of beats; and (C) superimpose the marker on the displayed ultrasonic image.

1. Ultrasonic Diagnostic Apparatus

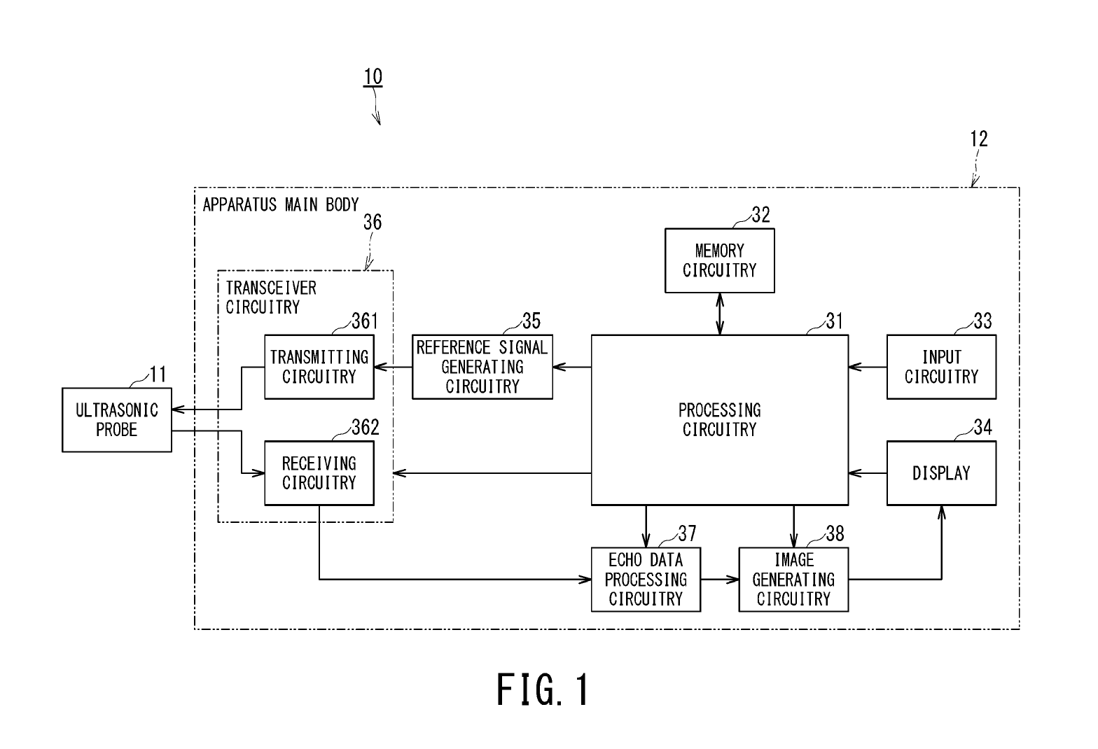

FIG. 1 is a schematic diagram showing a configuration of an ultrasonic diagnostic apparatus according to a present embodiment.

FIG. 1 shows the ultrasonic diagnostic apparatus 10 according to the present embodiment. The ultrasonic diagnostic apparatus 10 includes an ultrasonic probe 11 and an apparatus main body 12. The apparatus main body 12 is sometimes referred to as an ultrasonic diagnostic apparatus. In this case, the aforementioned ultrasonic diagnostic apparatus is connected to an ultrasonic probe provided outside of the ultrasonic diagnostic apparatus.

The ultrasonic probe 11 transmits and receives ultrasonic waves to and from an object (e.g., a patient). The ultrasonic probe 11 transmits and receives ultrasonic waves with its front surface being in contact with the surface of the object, and includes one- or two-dimensionally arranged minute transducers (piezoelectric elements), the number of which is M, at its distal end. The transducer is an electroacoustic transducer, and has functions of converting electric pulses into ultrasonic pulses (transmission ultrasonic waves) during transmission and converting ultrasonic reflected waves (reception ultrasonic waves) into an electric signal (received signal) during reception.

The ultrasonic probe 11 has a small and light-weight configuration, and is connected to the apparatus main body 12 through a cable. The ultrasonic probe 11 supports sector scanning, linear scanning, and convex scanning, any of which is freely selected in conformity with the diagnostic site.

The apparatus main body 12 includes a processor (e.g., processing circuitry) 31, a storage (e.g., memory circuitry) 32, an input device (e.g., input circuitry) 33, a display device (e.g., a display) 34, a reference signal generator (e.g., reference signal generating circuitry) 35, a transceiver (e.g., transceiver circuitry) 36, an echo data processor (e.g., echo data processing circuitry) 37, and an image generator (e.g., image generating circuitry) 38. A part of or the entire function of a digital circuit that constitutes the reference signal generator 35, the transceiver 36, the echo data processor 37 and the image generator 38 may be achieved through software that causes a processor to execute a program stored in a predetermined memory, in some cases. The description is herein made assuming that the functions of the reference signal generator 35, the transceiver 36, the echo data processor 37 and the image generator 38 are achieved through digital circuits.

The processing circuitry 31 reads various control programs stored in the memory circuitry 32 and performs various calculation, and integrally controls the processing operations in the elements 32 to 38.

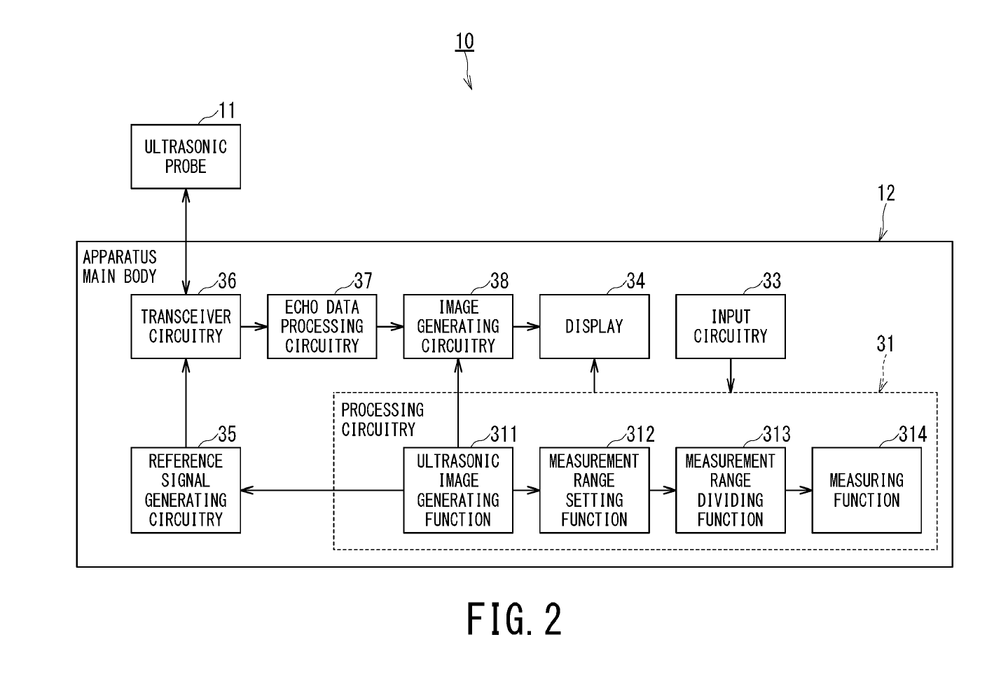

The processing circuitry 31 means any one of dedicated or general central processing unit (CPU) and a micro processor unit (MPU), an application specific integrated circuit (ASIC), and a programmable logic device. The programmable logic device may be, for example, any one of a simple programmable logic device (SPLD), a complex programmable logic device (CPLD), a field programmable gate array (FPGA) and the like. The processing circuitry 31 reads programs stored in the memory circuitry 32 or directly implemented in the processing circuitry 31 and executes these programs to achieve functions 311-314 shown in FIG. 2.

The processing circuitry 31 may be a single processing circuit or a combination of multiple processing circuits. In the latter case, the memory circuitry 32 includes multiple memory circuits each storing an element of a program, each of the multiple memory circuits is provided for each of the multiple circuits. Alternatively, the memory circuitry 32 includes a single memory circuit storing the program, the single memory circuit is provided for the multiple processing circuits.

The memory circuitry 32 includes semiconductor memory elements, such as RAM (random access memory) and flash memory, a hard disk, and an optical disk. The memory circuitry 32 may include portable media, such as USB (universal serial bus) memory and DVD (digital video disk). The memory circuitry 32 stores various processing programs used in the processing circuitry 31 (including not only application programs but also an OS (operating system)), data required to execute the programs, and ultrasonic images. The OS may include GUI (graphical user interface). The GUI uses many graphical items for displaying information for an operator on the display 34 and allows the operator to perform basic operations through the input circuitry 33.

The input circuitry 33 is a circuit that receives signals from input devices, such as a pointing device (such as mouse) and a keyboard, and is operable by the operator. It is herein assumed that the input device itself is included in the input circuitry 33. When the input device is operated by the operator, the input circuitry 33 generates an input signal according to the operation and outputs the signal to the processing circuitry 31. The apparatus main body 12 may include a touch panel where the input device is integrally configured with the display 34.

When the operator operates an end button or a freeze button of the input circuitry 33, transmission and reception of ultrasonic waves are finished and the ultrasonic diagnostic apparatus 10 is brought into a temporary stop state. The input circuitry 33 outputs a transmission condition set by the operator to the processing circuitry 31. The transmission condition is, for example, the central frequency of ultrasonic waves to be transmitted through the ultrasonic probe 11 to the object. The central frequency varies according to the scanning mode such as linear, convex or sector, the diagnostic target site of the object, the ultrasonic-diagnostic mode such as B mode, Doppler mode or color Doppler mode, the distance from the surface of the object to the diagnostic target site and the like.

The display 34, which includes a general display output device, such as a liquid crystal display or an OLED (organic light emitting diode) display, displays ultrasonic image data generated by the image generating circuitry 38 according to control by the processing circuitry 31.

The reference signal generating circuitry 35 generates, for example, continuous waves or square waves having a frequency substantially identical to the central frequency of the ultrasonic pulses, for the transceiver circuitry 36, according to a control signal from the processing circuitry 31.

The transceiver circuitry 36 causes the ultrasonic probe 11 to perform transmission and reception according to the control signal from the processing circuitry 31. The transceiver circuitry 36 includes: a transmitting circuitry 361 that generates a drive signal for causing the ultrasonic probe 11 to emit transmission ultrasonic waves from the ultrasonic probe 11; and a receiving circuitry 362 that applies phasing and addition to signals received from the ultrasonic probe 11.

The transmitting circuitry 361 includes a rate pulse generator, a transmission delay circuit, and a pulser, which are not shown. The rate pulse generator generates rate pulses that determine the repetitive period of transmission ultrasonic waves by frequency-dividing continuous waves or square waves supplied from the reference signal generating circuitry 35, and supplies the rate pulses to the transmission delay circuit. The transmission delay circuit, which includes independent delay circuits as many as the transducers used for transmission (M channels), provides the rate pulses with a delay time for converging the transmission ultrasonic waves to a predetermined depth to obtain a narrow beam width for transmission and a delay time for emitting the transmission ultrasonic waves in a predetermined direction, and supplies the rate pulses to the pulser. The pulser, which includes M-channel independent drive circuits, generates drive pulses for driving the transducers embedded in the ultrasonic probe 11, on the basis of the rate pulses.

The receiving circuitry 362 of the transceiver circuitry 36 includes a pre-amplifier, an A/D (analog to digital) conversion circuit, a reception delay circuit, and an adder circuit, which are not shown. The pre-amplifier, which has an M-channel configuration, amplifies weak signals converted as electric received signals by the transducers, and secures a sufficient S/N. The M-channel received signals amplified to have predetermined intensities by the pre-amplifier are converted into digital signals by the A/D conversion circuit, and transmitted to the reception delay circuit. The reception delay circuit provides each of the M-channel received signals output from the A/D conversion circuit with the delay time for convergence to converge the ultrasonic reflected waves from the predetermined depth and the delay time for deflection to set a receiving directionality with respect to a predetermined direction. The adder circuit applies phasing and addition to signals received from the reception delay circuit (adds up the phases of signals received in the predetermined direction).

The echo data processing circuitry 37 applies a process for generating an ultrasonic image to the echo data input from the receiving circuitry 362 according to the control signal from the processing circuitry 31. For example, the echo data processing circuitry 37 performs a process such as a B-mode process or a Doppler process, the B-mode process including a logarithmic compression process and an envelope detection process, the Doppler process including an orthogonal detection process and a filtering process.

The image generating circuitry 38 causes a scan converter to scan and convert the data received from the echo data processing circuitry 37 to generate ultrasonic image data, according to the control signal from the processing circuitry 31. The image generating circuitry 38 then causes the display 34 to display an ultrasonic image based on the ultrasonic image data. The ultrasonic images include, for example a B-mode image which is sectional information at a given time (frame), and an M-mode image and a Doppler spectrum image, in which temporal variation over a given time period is imaged.

Subsequently, the functions of the ultrasonic diagnostic apparatus 10 according to the present embodiment are described.

FIG. 2 is a block diagram showing functions of the ultrasonic diagnostic apparatus 10 according to the present embodiment.

Execution of programs by the processing circuitry 31 allows the ultrasonic diagnostic apparatus 10 to function as an ultrasonic image generating function 311, a measurement range setting function 312, a measurement range dividing function 313, and a measuring function 314. The description is made exemplifying a case where the functions 311 to 314 operate as software. Alternatively, some or all of the functions 311 to 314 may be provided as hardware for the ultrasonic diagnostic apparatus 10.

The ultrasonic image generating function 311 is a function of controlling the operation of the ultrasonic probe 11 through the reference signal generating circuitry 35 to perform scanning, and causing the image generating circuitry 38 to generate an ultrasonic image. The ultrasonic image generating function 311 generates, as the ultrasonic image, at least an ultrasonic image (such as the M-mode image or the Doppler spectrum image) where temporal variation over the given time period is imaged. The ultrasonic image generating function 311 causes the display 34 to display the ultrasonic image.

The B-mode image is an amplitude distribution image of luminance values converted from the reflective strengths (amplitudes) obtained by transmitting and receiving ultrasonic beams along rasters (scanning lines) in the array direction (azimuth direction). Furthermore, B-mode images are generated over a given time period.

The Doppler spectrum image is an image representing flow velocities (flow velocity transition) obtained over a given time period. For example, the Doppler spectrum image represents the flow velocity transition at a position of the sample volume (SV) set on the B-mode image.

The M-mode image is an image representing amplitudes (amplitude transition) obtained by transmitting and receiving the ultrasonic beam to and from one raster in the array direction over a given time period. For example, the M-mode image represents the amplitude transition at the position of a raster that is set on the B-mode image.

A case is hereinafter described where the ultrasonic image generating function 311 generates the Doppler spectrum image as the ultrasonic image where temporal variation over the given time period is imaged.

The measurement range setting function 312 is a function of setting a measurement range (measurement time period) for a measurement target, such as a heart rate, on the basis of the Doppler spectrum image generated by the ultrasonic image generating function 311. The measurement range setting function 312 sets two telediastolic times in the cardiac sequence to thereby set the measurement range between the set two times. The end-diastole is determined on the basis of R waves in an ECG signal (electrocardiographic signal).

The measurement range dividing function 313 is a function of generating markers (or a marker) for dividing the measurement range set by the measurement range setting function 312, at substantially regular intervals, the number of markers according to the numerically set number of beats, and superimposing the markers on the displayed ultrasonic image. Preferably, the number of markers according to the numerically set number of beats is “n−1” in a case where the numerically set number of beats is “n” (n: an integer at least two). However, the number is not limited to this case.

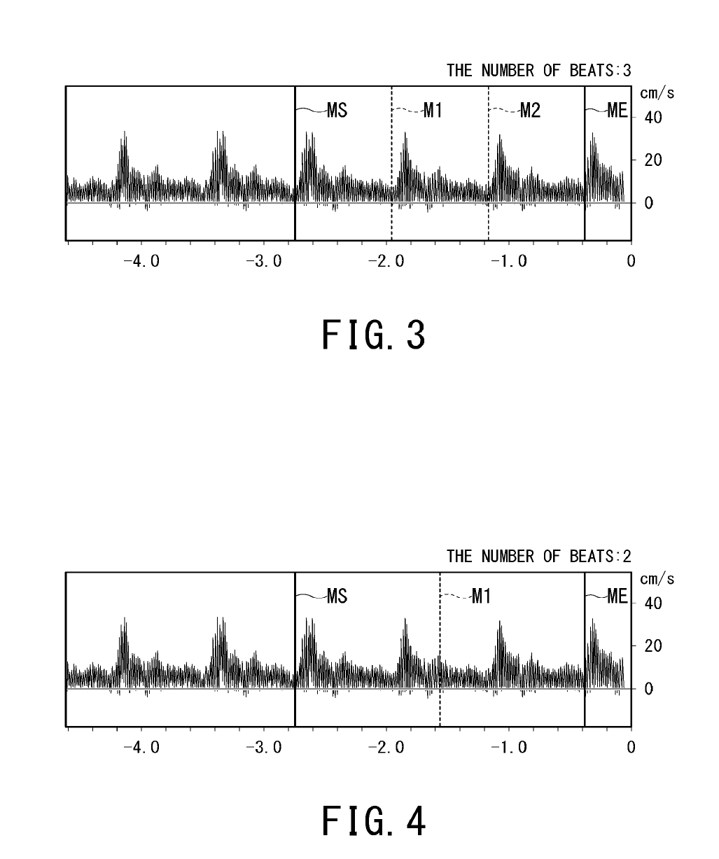

Each of FIGS. 3 to 7 is a diagram to illustrate an example of dividing the measurement range.

Each of FIGS. 3 to 5 and 7 schematically shows a Doppler spectrum image that represents Doppler waveforms. First, in the Doppler mode, a Doppler spectrum image in a time period including heartbeats is generated and the state is brought into the freeze state. When a measurement mode for the heart rate is activated, measurement range markers MS and ME that represent two times (a start time and an end time) based on R waves are displayed on the Doppler spectrum image by the operator, and the measurement range markers MS and ME are manually, laterally slid as necessary to thereby designate two times. FIG. 6 schematically shows the M-mode image. First, in the M mode, an M-mode image in a time period including heartbeats is generated and the state is brought into the freeze state. When the measurement mode for the heart rate is activated, measurement range markers MS and ME that represent two times based on R waves are displayed on the M-mode image by the operator, and the measurement range markers MS and ME are manually, laterally slid as necessary to thereby designate two times.

Here, the numerical value of the number of beats is set irrespective of the measurement range indicated by the measurement range markers MS and ME. The numerical value of the number of beats is set as a default or set by changing the default value. In the case where the numerically set number of beats is “n”, “n−1” dividing times (or a dividing time) for equally dividing the measurement range between the set two times into “n” are calculated, and the “n−1” dividing markers (or a dividing marker) that represent dividing times are aligned and displayed on the Doppler spectrum image (or M-mode image).

More specifically, FIG. 3 shows a Doppler spectrum image and measurement range markers MS and ME in the case where the numerically set number of beats is three (n=3). As shown in FIG. 3, in the case where the numerically set number of beats is three, two dividing times for equally dividing the measurement range between the set two times into three are calculated, and the two dividing markers M1 and M2 that represent dividing times are aligned and displayed on the Doppler spectrum image. In FIG. 3, the markers MS, ME, M1 and M2 are displayed as lines (solid lines and broken lines) representing the times, on the Doppler spectrum image. Some or all of the markers MS, ME, M1 and M2 may be displayed as pointers that represent times on the Doppler spectrum image. According to such display, it is possible for the operator to intuitively, visually identify the number of beats numerically set irrespective of the measurement range, on the Doppler spectrum image.

In the example shown in FIG. 3, the numerically set number of beats “3” coincides with the number of beats in the measurement range between the set two times “3”. Consequently, if the heartbeat is not disordered, the dividing markers M1 and M2 substantially coincide with two end-diastoles between the measurement range markers MS and ME.

FIG. 4 shows a Doppler spectrum image and measurement range markers MS and ME in the case where the numerically set number of beats is two (n=2). As shown in FIG. 4, in the case where the numerically set number of beats is two, one dividing time for equally dividing the measurement range between the set two times into two are calculated, and the one dividing marker Ml that represents a dividing time is aligned and displayed on the Doppler spectrum image. According to such display, it is possible for the operator to intuitively, visually identify the number of beats numerically set irrespective of the measurement range, on the Doppler spectrum image.

In the example shown in FIG. 4, the numerically set number of beats “2” does not coincide with the number of beats in the measurement range between the set two times “3”. Consequently, the dividing marker M1 does not coincide with two end-diastoles between the measurement range markers MS and ME.

Therefore, in comparison between FIGS. 3 and 4, it is possible for the operator to intuitively, visually identify the inconsistency between the number of beats numerically set irrespective of the measurement range and the number of beats in the measurement range between the two times set on the Doppler spectrum image.

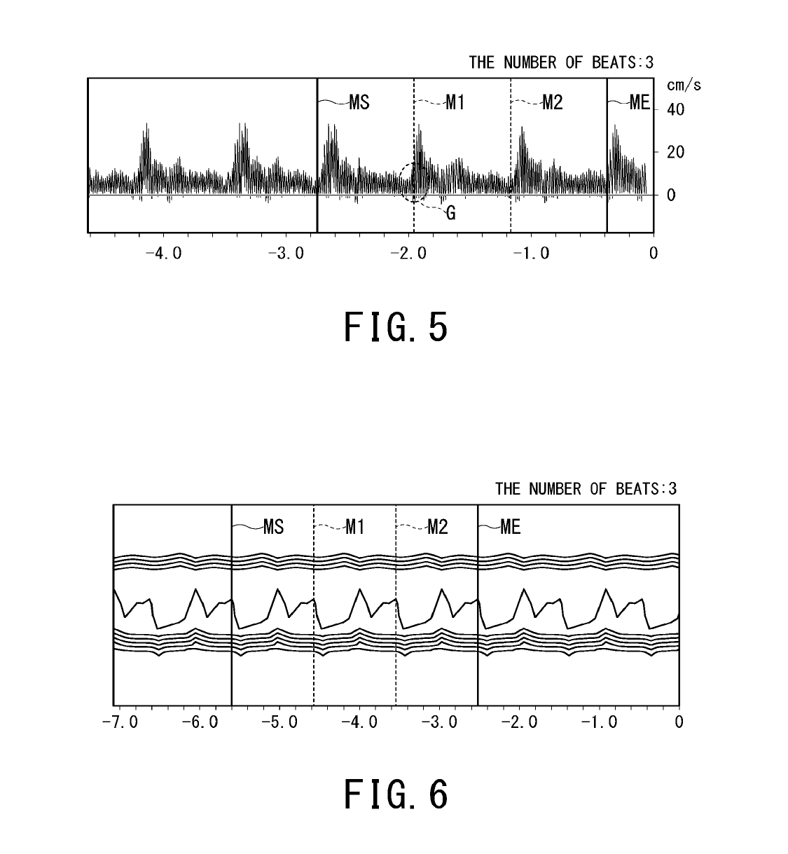

FIG. 5 shows a Doppler spectrum image and measurement range markers MS and ME in the case where the numerically set number of beats is three (n=3). As shown in FIG. 5, in the case where the numerically set number of beats is three, two dividing times for equally dividing the measurement range between the set two times into three are calculated, and the two dividing markers M1 and M2 that represent dividing times are aligned and displayed on the Doppler spectrum image. According to such display, it is possible for the operator to intuitively, visually identify the number of beats numerically set irrespective of the measurement range, on the Doppler spectrum image.

In the example shown in FIG. 5, the numerically set number of beats “3” coincides with the number of beats in the measurement range between the set two times “3”. There is however a disorder (abnormal cardiac rhythm) as indicated by G. Consequently, the dividing markers M1 and M2 do not coincide with two end-diastoles between the measurement range markers MS and ME.

Therefore, in comparison between FIGS. 3 and 5, it is possible for the operator to intuitively, visually identify the disorder (abnormal cardiac rhythm) between the measurement range markers MS and ME.

In case an abnormal cardiac rhythm is automatically recognized in the measurement range, the recognition may be notified to the operator. This is because a waveform including an abnormal cardiac rhythm during measurement using the measurement range is intended to be excluded from the measurement target. For example, in case the flow velocities at positions where a trace line tracing a Doppler waveform crosses the dividing markers M1 and M2 exceed a threshold, presence of an abnormal cardiac rhythm is identified. When the presence of an abnormal cardiac rhythm is identified, heartbeats where the abnormal cardiac rhythm occurs are automatically loop-reproduced.

FIG. 6 shows an M-mode image and measurement range markers MS and ME in the case where the numerically set number of beats is three (n=3). As shown in FIG. 3, in the case where the numerically set number of beats is three, two dividing times for equally dividing the measurement range between the set two times into three are calculated, and the two dividing markers M1 and M2 that represent dividing times are aligned and displayed on the M-mode image. According to such display, it is possible for the operator to intuitively, visually identify the number of beats numerically set irrespective of the measurement range on the M-mode image.

In the example shown in FIG. 6, the numerically set number of beats “3” coincides with the number of beats in the measurement range between the set two times “3”. Consequently, if the heartbeat is not disordered, the dividing markers M1 and M2 substantially coincide with two end-diastoles between the measurement range markers MS and ME.

Therefore, according to FIG. 6, it is possible for the operator to intuitively, visually identify the inconsistency between the number of beats numerically set irrespective of the measurement range and the number of beats in the measurement range between the set two times set on the M-mode image.

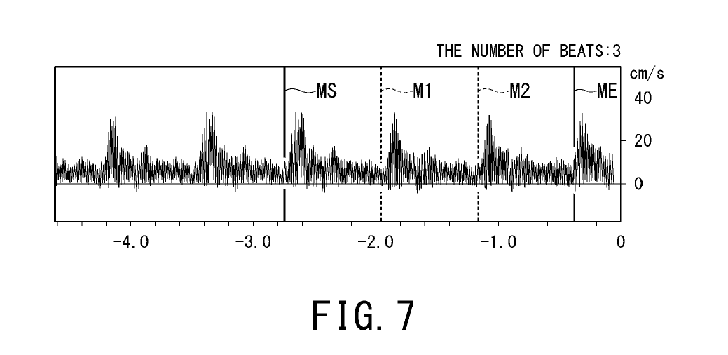

FIG. 7 shows a variation example of the display of FIG. 3. As shown in FIG. 7, pixels around which pixels representing a Doppler waveform exist among the pixels indicating the measurement range markers MS and ME and the dividing markers M1 and M2 are set to have a luminance value representing the Doppler waveform. That is, when a part of the markers MS, ME, M1 and M2 is disposed around a Doppler spectrum, this part is set to be hidden (or translucent).

Consequently, it is possible for the operator to preferentially, visually identify the Doppler spectrum image without interference by the display of the measurement range markers MS and ME and the dividing markers M1 and M2. Returning to the description on FIG. 2, the measuring function 314 is a function of performing measurement in the measurement range set by the measurement range setting function 312. The measuring function 314 measures a heart rate (BPM: beats per minute) [bpm] based on the number of beats in the measurement range set by the measurement range setting function 312. The measured numerical value is displayed on the display 34.

The measuring function 314 may measure the maximum flow velocity in the Doppler spectrum image by tracing the maximum flow velocity in the measurement range set by the measurement range setting function 312. Alternatively, the flow velocity range of the Doppler spectrum image may be measured on the basis of the variation in flow velocity in the measurement range set by the measurement range setting function 312.

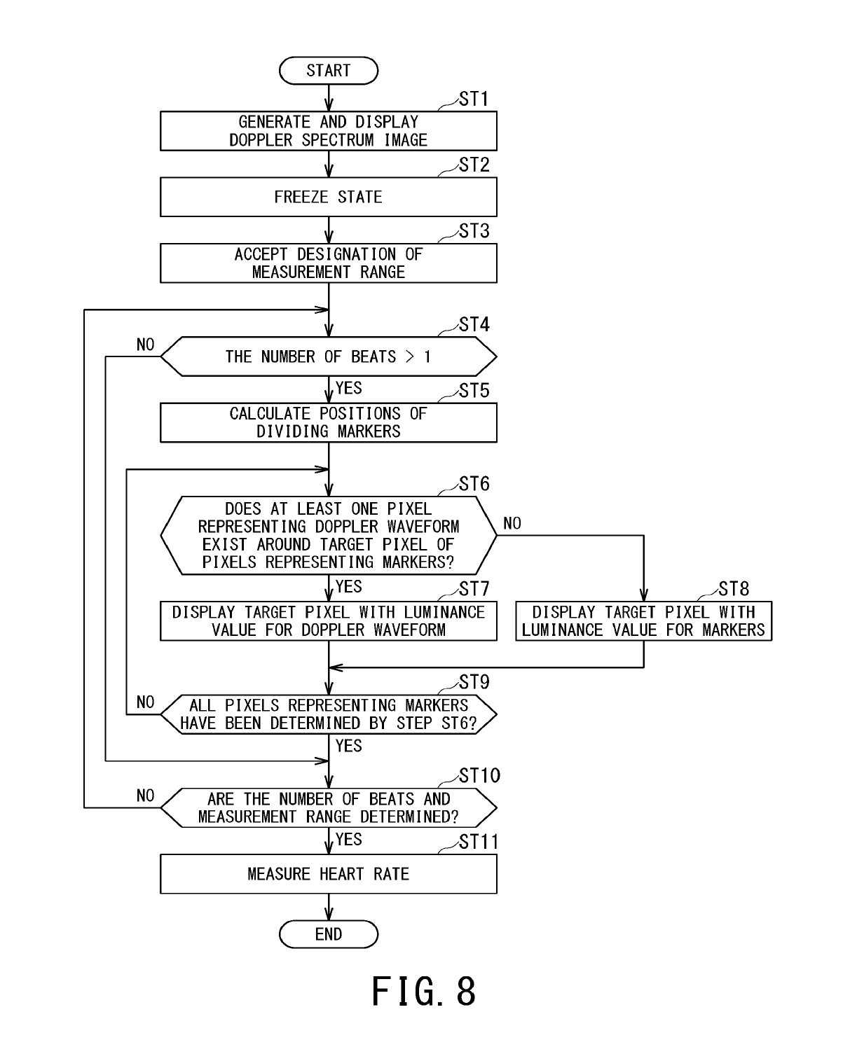

Subsequently, referring to FIGS. 1 and 8, the operation of the ultrasonic diagnostic apparatus 10 according to the present embodiment is described.

FIG. 8 is a flowchart showing operations of the ultrasonic diagnostic apparatus 10 according to the present embodiment.

The processing circuitry 31 controls the reference signal generating circuitry 35 and the like to execute scanning, generates a Doppler spectrum image in a mode for generating an ultrasonic image where temporal variation over a given time period is imaged, for example, the Doppler mode, and displays the Doppler spectrum image on the display 34 (step ST1). The ultrasonic image generated in step ST1 may be an M-mode image generated in the M mode.

After transition to the mode for measuring the heart rate and the like, the processing circuitry 31 brings the Doppler mode into the freeze state (step ST2), and accepts designation of the measurement range (measurement time period) for the measurement target, such as the heart rate, from the operator (step ST3).

The processing circuitry 31 determines whether the numerically set number of beats is higher than one (n>1) or not (step ST4). In step ST4, the processing circuitry 31 uses, as the numerically set number of beats, a number set as the default or a number set by changing the default value according to NO in step ST10.

In the case of YES in the determination in step ST4, that is, in the case where the numerically set number of beats is determined to be higher than one, the processing circuitry 31 calculates “n−1” dividing times for equally dividing the measurement range designated in step ST3 into “n”, and calculates the positions (times) of the “n−1” dividing markers representing the dividing times (step ST5).

The processing circuitry 31 determines whether at least one pixel representing a Doppler waveform exists (e.g., within 10 pixels) or not around a target pixel of pixels representing the dividing markers and the measurement range markers (step ST6). In the case of YES in the determination in step ST6, that is, in the case where at least one pixel representing the Doppler waveform exists around the target pixel of the pixels representing the dividing markers and the measurement range markers, the processing circuitry 31 displays the target pixel with the luminance value of the Doppler waveform (step ST7).

On the contrary, in the case of NO in the determination in step ST6, that is, in the case where no pixel representing the Doppler waveform exists around the target pixel of the pixels representing the dividing markers and the measurement range markers, the processing circuitry 31 displays the target pixel with the luminance value for the dividing markers and the measurement range markers (step ST8).

The processing circuitry 31 determines whether or not the processing circuitry 31 has performed the determination in step ST6 for all the pixels representing the dividing markers and the measurement range markers (step ST9). In the case of YES in the determination in step ST9, that is, in the case where it is determined that the determination in step ST6 has been performed for all the pixels representing the dividing markers and the measurement range markers, the processing circuitry 31 determines whether or not to finally determine the numerically set number of beats and the measurement range designated in step ST3 (step ST10).

In the case of YES in the determination in step ST10, that is, in the case where it is determined to finally determine the numerically set heart rate and the measurement range designated in step ST3, the processing circuitry 31 measures the heart rate on the basis of the number of beats and the measurement range finally determined in step ST10 (step ST11).

On the contrary, in the case of NO in the determination in step ST10, that is, in the case where it is determined not to finally determine the numerically set heart rate and the measurement range designated in step ST3 and to change the heart rate or the measurement range instead, the processing circuitry 31 performs the determination in step ST4 for the changed heart rate and measurement range.

For example, in the case of NO in the determination in step ST10, that is, in the case where the measurement range marker ME (shown in FIG. 3) is slid to the left through an operation by the operator, the dividing markers M1 and M2 (shown in FIG. 3) are slid to the left while the intervals for the equally divided three are maintained in the measurement range (step ST5).

In the case of NO in the determination in step ST4, that is, in the case where it is determined that the numerically set number of beats is one or lower, the processing circuitry 31 determines whether or not to finally determine the numerically set heart rate and the measurement range designated in ST3 (step ST10).

In the case of NO in the determination in step ST9, that is, in the case where it is determined that the determination in step ST6 has been performed for not all the pixels representing the dividing marker and the measurement range marker, the processing circuitry 31 returns the processing to the determination in step ST6 for the next one of the pixels representing the dividing markers and the measurement range markers.

The markers MS, ME, M1 and M2 are thus displayed in the mode shown in FIG. 7 where all the pixels representing the dividing markers and the measurement range markers are displayed with the luminance value of the Doppler waveform or the marker. In FIG. 8, when all the pixels representing the dividing markers and the measurement range markers are displayed with the luminance value of the marker, the markers MS, ME, M1 and M2 are displayed in the mode shown in FIGS. 3 and 4.

The ultrasonic diagnostic apparatus 10 thus displays the marker(s) corresponding to the numerically set number of beats on the ultrasonic image. Consequently, it is possible for the operator to intuitively, visually identify an erroneous setting of the number of beats and the measurement range.

2. Medical Image Processing Apparatus

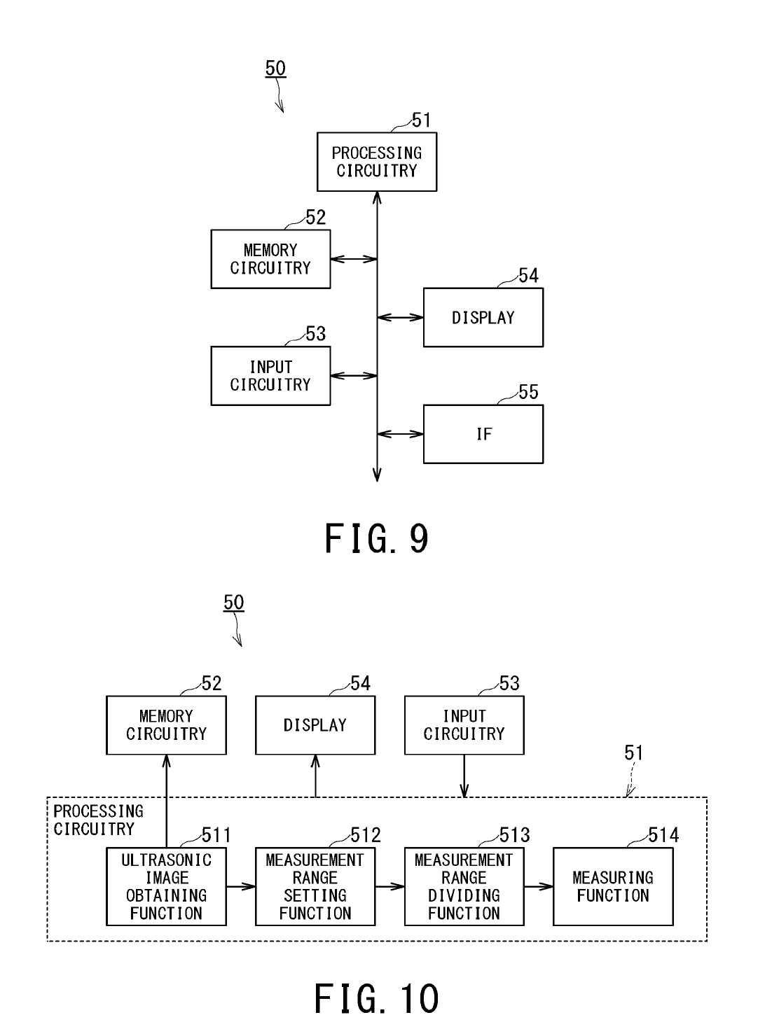

FIG. 9 is a schematic diagram showing a configuration of a medical image processing apparatus according to a present embodiment.

FIG. 9 shows the medical image processing apparatus 50 according to the present embodiment. The medical image processing apparatus 50 is any of a medical image management apparatus (image server), a workstation, a reading terminal and the like, which are not shown, and is provided in a medical imaging system to which this apparatus is connected via a network. Alternatively, the medical image processing apparatus 50 may be an offline apparatus.

The medical image processing apparatus 50 includes a processing unit (e.g., processing circuitry) 51, a storage (e.g., memory circuitry) 52, an input device (e.g., input circuitry) 53, a display device (e.g., a display) 54, and a communication unit (e.g., IF (interface)) 55.

The processing circuitry 51 has a configuration equivalent to that of the processing circuitry 31 shown in FIG. 1. The processing circuitry 51 reads various control programs stored in the memory circuitry 52 and performs various calculation, and integrally controls the processing operation in the elements 52 to 55.

The memory circuitry 52 has a configuration equivalent to that of the memory circuitry 32 shown in FIG. 1. The memory circuitry 52 stores various processing programs used in the processing circuitry 51, data required to execute the programs, and medical images, such as ultrasonic images, obtained via the IF 55. The OS may include GUI. The GUI uses many graphical items for displaying information for an operator on the display 54 and allows the operator to perform basic operations through the input circuitry 53.

The input circuitry 53 has a configuration equivalent to that of the input circuitry 33 shown in FIG. 1. When the input device is operated by the operator, the input circuitry 53 generates an input signal according to the operation and outputs the signal to the processing circuitry 51. The medical image processing apparatus 50 may include a touch panel where the input device is integrally configured with the display 54.

The display 54 has a configuration equivalent to that of the display 34 shown in FIG. 1. The display 54 displays image data generated according to the control by the processing circuitry 51.

The IF 55 includes a connector in conformity with parallel connection specifications and serial connection specifications. The IF 55 performs communication control according to the corresponding specifications, and has a function of connection to a network via a telephone line. This function connects the medical image processing apparatus 50 to the network.

Subsequently, the functions of the medical image processing apparatus 50 according to the present embodiment are described.

FIG. 10 is a block diagram showing functions of the medical image processing apparatus 50 according to the present embodiment.

Execution of programs by the processing circuitry 51 allows the medical image processing apparatus 50 to function as an ultrasonic image obtaining function 511, a measurement range setting function 512, a measurement range dividing function 513, and a measuring function 514. The description is made exemplifying a case where the functions 511 to 514 operate as software. Alternatively, some or all of the functions 511 to 514 may be provided as hardware for the medical image processing apparatus 50.

The ultrasonic image obtaining function 511 is a function of obtaining (reading), as the ultrasonic image, at least an ultrasonic image where temporal variation over a given time period is imaged (e.g., an M-mode image or a Doppler spectrum image), from the memory circuitry 52. The ultrasonic image obtaining function 511 causes the display 54 to display the ultrasonic image.

The measurement range setting function 512, the measurement range dividing function 513 and the measuring function 514 function in manners analogous to those of the measurement range setting function 312, the measurement range dividing function 313 and the measuring function 314 shown in FIG. 2.

The operations of the medical image processing apparatus 50 are analogous to the operations in steps ST3 to ST11 in the ultrasonic diagnostic apparatus 10 shown in FIG. 8. Consequently, description of the operations is omitted.

The medical image processing apparatus 50 displays the marker(s) corresponding to the numerically set number of beats on the ultrasonic image. Consequently, it is possible for the operator to intuitively, visually identify an erroneous setting of the number of beats and the measurement range.

According to at least one of the ultrasonic diagnostic apparatus and the medical image processing apparatus of the embodiments described above, it is possible for the operator to intuitively, visually identify an erroneous setting on the number of beats and the measurement range and to reduce the test time.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus, comprising:
transceiver circuitry configured to transmit, to an ultrasonic probe, a transmission signal for transmitting ultrasonic waves, and to receive a received signal based on the ultrasonic waves;
image generating circuitry configured to generate an ultrasonic image where temporal variation over a given time period is imaged, based on the received signal, and to display the ultrasonic image to a display; and
processing circuitry configured to
(A) set a measurement range on the ultrasonic image,
(B) generate a marker for dividing the measurement range, at substantially equal intervals, the number of the marker according to the numerically number of beats, and
(C) superimpose the marker on the displayed ultrasonic image.

2. The ultrasonic diagnostic apparatus according to claim 1,
wherein the processing circuitry is configured to display the marker on the ultrasonic image as a line representing a time.

3. The ultrasonic diagnostic apparatus according to claim 2,
wherein the processing circuitry is configured to set, when a part of the line is disposed around a waveform of a Doppler spectrum image, the part to be hidden or translucent.

4. The ultrasonic diagnostic apparatus according to claim 1,
wherein the processing circuitry is configured to set, in a case where the number of beats is “n” (n: an integer at least two), the number of the marker according to the number of beats to “n−1”.

5. The ultrasonic diagnostic apparatus according to claim 1,
wherein the image generating circuitry is configured to generate a Doppler spectrum image as the ultrasonic image.

6. The ultrasonic diagnostic apparatus according to claim 1,
wherein the image generating circuitry is configured to generate an M-mode image as the ultrasonic image.

7. The ultrasonic diagnostic apparatus according to claim 1,
wherein the processing circuitry is configured to further measure a heart rate based on the number of beats and the measurement range.

8. The ultrasonic diagnostic apparatus according to claim 1,
wherein the processing circuitry is configured to further measure a maximum flow velocity by tracing a maximum flow velocity in the measurement range on the Doppler spectrum image as the ultrasonic image, based on the measurement range.

9. The ultrasonic diagnostic apparatus according to claim 1,
wherein the processing circuitry is configured to further measure a flow velocity range by variation in flow velocity in the measurement range on the Doppler spectrum image as the ultrasonic image, based on the measurement range.

10. The ultrasonic diagnostic apparatus according to claim 1,
wherein the processing circuitry is configured to issue, when a flow velocity at a position where a Doppler waveform of the Doppler spectrum image as the ultrasonic image crosses the marker exceeds a threshold, a notification of the exceeding.

11. A medical image processing apparatus, comprising a processing circuitry configured to:
obtain an ultrasonic image where temporal variation over a given time period is imaged, and display the ultrasonic image to a display;
set a measurement range on the ultrasonic image;
generate a marker for dividing the measurement range, at substantially equal intervals, the number of the marker according to the numerically number of beats; and
superimpose the marker on the displayed ultrasonic image.
